(19) United States
(12) Patent Application Publication
(10) Pub. No.: US 2025/0277088 A1
(43) Pub. Date: Sep. 4, 2025

(54) BIOCOMPATIBLE HYDROGEL COMPRISING COMPONENT CONTAINING HYALURONIC ACID, POLYETHYLENE GLYCOL AND SILICONE

(71) Applicant: KYUNGPOOK NATIONAL UNIVERSITY INDUSTRY-ACADEMIC COOPERATION FOUNDATION, Buk-gu, Daegu (KR)

(72) Inventors: Jeong Soo YOO, Buk-gu, Daegu (KR); Wan Wook KIM, Buk-gu, Daegu (KR); Seong Hwan CHO, Buk-gu, Daegu (KR)

(21) Appl. No.: 18/272,951

(22) PCT Filed: Jan. 18, 2022
(86) PCT No.: PCT/KR2022/000933

§ 371 Date: Jul. 18, 2023

(30) Foreign Application Priority Data
Jan. 18, 2021 (KR) 10-2021-0006872

(51) Int. Cl. C08J 3/075; A61L 26/00; C08J 3/24; C08L 5/08; C08L 71/02; C08L 83/04

(52) U.S. Cl. CPC C08J 3/075; A61L 26/0052; A61L 26/008; C08J 3/246; C08L 5/08; C08L 71/02; C08L 83/04; C08L 2203/02; C08L 2312/06

(57) ABSTRACT

The present invention relates to a biocompatible hydrogel comprising a component containing hyaluronic acid, polyethylene glycol and a silicone and, more specifically, to a biocompatible hydrogel, a preparation method thereof, and the use thereof, the biocompatible hydrogel prepared by inducing intermolecular and/or intramolecular cross-linking of a component containing hyaluronic acid, polyethylene glycol and a silicone, by irradiating radiation without adding a cross-linking agent and the like or without using a reactor.

### TECHNICAL FIELD

The present application claims priority to Korean Patent Application No. 2021-0006872 filed on Jan. 18, 2021, and the entire specification thereof is incorporated herein by reference.

The present invention relates to a biocompatible hydrogel containing hyaluronic acid, polyethylene glycol and a silicone-containing component, and, more specifically, to a biocompatible hydrogel prepared by inducing intermolecular and/or intramolecular cross-linking of hyaluronic acid, polyethylene glycol and a silicone-containing component only by irradiation without adding a reactive group or a chemical cross-linking agent, a method for producing same, and use thereof.

### BACKGROUND ART

Recently, hydrogels have attracted a lot of attention in the medical field and are expected to be widely used in medical fillers, release systems for physiologically active substances, and organ/tissue regeneration using three-dimensional structures.

These hydrogels have generally been prepared by cross-linking by adding chemicals such as a cross-linking agent and/or a curing agent to a polymer material. However, since the cross-linking agent and/or curing agent itself used in the cross-linking reaction is harmful to the living body, there is a problem in that a hydrogel prepared by using such a cross-linking agent and/or curing agent may cause harmful effects when used in a living body. In particular, these hydrogels are unsuitable for use as medical and pharmaceutical materials, such as wound dressings, drug delivery carriers, contact lenses, cartilage, intestinal adhesion inhibitors, and the like. In addition, when a cross-linking agent and/or curing agent is used, the residual cross-linking agent and/or curing agent in the hydrogel must be removed after preparing the hydrogel, thereby causing the problems that the manufacturing process is complicated, and the manufacturing cost is increased.

Accordingly, efforts are being made to prepare a polymer-derived hydrogel without using a cross-linking agent and/or a curing agent, and, as a result of these efforts, the production of a hydrogel by irradiating a synthetic polymer has been reported.

However, hydrogels derived from synthetic polymers are not suitable for medical or pharmaceutical use in terms of biocompatibility and biodegradability. Thus, there is a need for the development of hydrogels formed only by intra-molecular or inter-molecular cross-linking of biocompatible molecules, without using cross-linking agents, curing agents, organic solvents, and the like.

On the other hand, hyaluronic acid is a type of polysaccharide in which repeating units consisting of N-acetyl-glucosamine and D-glucuronic acid are linearly connected, and is a biopolymer material. Since it was first isolated from the liquid filling the eyes of animals, it is known that hyaluronic acid is present in large amounts in the placenta of animals, synovial fluid of joints, pleural fluid, skin and rooster crests, and is also produced in genus Streptococcus microorganisms Streptococcus equi, Streptococcus zooepidemecus, and the like.

Since hyaluronic acid has excellent biocompatibility and high viscoelasticity in a solution state, it is widely used not only for cosmetic applications such as cosmetic additives, but also for various medical applications such as ophthalmic surgical aids, joint function improving agents, drug delivery materials and eye drops. However, hyaluronic acid itself is easily degraded in vivo or under acidic or alkaline conditions, and its use is very limited. Thus, it is common to adda chemical cross-linking agent to the production of hyaluronic acid-based hydrogels (see WO 2013/055832).

In particular, it is well known in the art that biocompatible polymers such as carboxymethylcellulose, methylcellulose, hydroxyethylcellulose and carboxymethylstarch can form gels by irradiation (see Nuclear Instruments and Methods in Physics Research B 208 (2003), pp. 320-324; Carbohydrate Polymers 112 (2014), pp. 412-415; and Nuclear Instruments and Methods in Physics Research B 211 (2003), pp. 533-544). On the other hand, in the case of hyaluronic acid, a degradation reaction such as a decrease in molecular weight and a decrease in viscosity is easily caused by irradiation (see Korean Laid-open Patent Publication No. 2008-0086016). Therefore, hyaluronic acid-based hydrogels prepared through irradiation, i.e., hyaluronic acid-based hydrogels prepared only by irradiation without adding chemical cross-linking agents and organic chemicals, have not yet been provided.

Korean Patent No. 2070878 discloses a method for producing a Merkgel for filler treatment, which comprises preparing a bulk hydrogel by cross-linking hyaluronic acid by irradiating 0.5 to 5 kGy of an electron-beam to 10 to 20 w/v % of a hyaluronic acid aqueous solution for 30 seconds to 5 minutes. However, there is a limitation in that, since hyaluronic acid is capable of absorbing moisture up to several times its own weight, it is very difficult to prepare 10 to 20 w/v % of a hyaluronic acid aqueous solution in a conventional manufacturing facility, and a hydrogel having various physical properties by using hyaluronic acid cannot be produced.

On the other hand, silicon is a biocompatible polymeric material that is stable to heat, has excellent oxygen permeability and is transparent and non-toxic. Because of these characteristics, silicone-containing compounds have been used as catheters, discharge tubes, pacemakers, membrane oxygenators and biomaterials such as ear and nose implants, and for various purposes ranging from contact lenses, medical devices such as implants, and elastomers. In particular, in cosmetics, a silicone-containing component is not only widely used to improve application properties of cosmetics but also serves as a skin lubricant to add gloss without stickiness. It also forms a thin layer after being applied to the skin to prevent evaporation of moisture.

As such, if a hydrogel which comprises both hyaluronic acid and a silicone-containing component and exhibits high biocompatibility and various advantages can be prepared without using a chemical cross-linking agent or an organic solvent, it is expected to be very useful in the development of pharmaceuticals, medical devices, quasi-drugs, cosmetics and skin care products.

### SUMMARY OF INVENTION

### Technical Problem

Accordingly, the inventors of the present invention conducted extensive research to develop a biocompatible hydrogel based on hyaluronic acid and silicone produced only by irradiation without using cross-linking agents or organic chemicals, and they have confirmed that a hydrogel which comprises hyaluronic acid, polyethylene glycol and a silicone-containing component and exhibits various physical properties can be prepared by using polyethylene glycol, another biocompatible polymer, together, under specific conditions for preparation, and have completed the present invention.

Therefore, an object of the present invention is to provide a hydrogel formed only by inter-molecular cross-linking, intra-molecular cross-linking, or intermolecular and intra-molecular cross-linking of hyaluronic acid, polyethylene glycol (PEG) and a silicone-containing component.

Another object of the present invention is to provide a method for preparing a hydrogel formed only by inter-molecular cross-linking, intra-molecular cross-linking, or intermolecular and intra-molecular cross-linking of hyaluronic acid, polyethylene glycol (PEG) and a silicone-containing component, the method comprising the following steps: (a) adding hyaluronic acid, polyethylene glycol and a silicone-containing component to water, thereby preparing a solution; and (b) irradiating radiation to the solution produced in step (a) to induce cross-linking of the added substances.

Another object of the present invention is to provide a cell delivery system, drug delivery system, anti-adhesion agent, cell support, dental filler, orthopedic filler, wound dressing or dermal filler, comprising the hydrogel.

Another object of the present invention is to provide a composition for application to the wounded area of skin, comprising the hydrogel as an active ingredient.

In addition, it is to provide a composition for application to the wounded area of skin, consisting of the hydrogel.

In addition, it is to provide a composition for application to the wounded area of skin, consisting essentially of the hydrogel.

Another object of the present invention is to provide use of the hydrogel for preparing an agent for application to the wounded area of skin.

Another object of the present invention is to provide a method of treating the wounded area of skin by applying an effective amount of a composition comprising the hydrogel as an active ingredient to the skin of a subject in need thereof.

### Solution to Problem

In order to achieve the above-mentioned object of the present invention, the present invention provides a hydrogel formed only by inter-molecular cross-linking, intra-molecular cross-linking, or intermolecular and intra-molecular cross-linking of hyaluronic acid, polyethylene glycol (PEG) and a silicone-containing component.

In order to achieve another object of the present invention, the present invention provides a method for preparing a hydrogel formed only by inter-molecular cross-linking, intra-molecular cross-linking, or intermolecular and intra-molecular cross-linking of hyaluronic acid, polyethylene glycol (PEG) and a silicone-containing component, the method comprising the following steps: (a) adding hyaluronic acid, polyethylene glycol and a silicone-containing component to water, thereby preparing a solution; and (b) irradiating radiation to the solution produced in step (a) to induce cross-linking of the added substances.

In order to achieve another object of the present invention, the present invention provides a cell delivery system, drug delivery system, anti-adhesion agent, cell support, dental filler, orthopedic filler, wound dressing or dermal filler, comprising the hydrogel.

In order to achieve another object of the present invention, the present invention provides a composition for application to the wounded area of skin, comprising the hydrogel as an active ingredient.

In addition, the present invention provides a composition for application to the wounded area of skin, consisting of the hydrogel.

In addition, the present invention provides a composition for application to the wounded area of skin, consisting essentially of the hydrogel.

In order to achieve another object of the present invention, the present invention provides use of the hydrogel for preparing an agent for application to the wounded area of skin.

In order to achieve another object of the present invention, the present invention provides a method of treating the wounded area of skin by applying an effective amount of a composition comprising the hydrogel as an active ingredient to the skin of a subject in need thereof.

Hereinafter, the present invention will be described in detail.

The present invention provides a hydrogel formed only by inter-molecular cross-linking, intra-molecular cross-linking, or intermolecular and intra-molecular cross-linking of hyaluronic acid, polyethylene glycol (PEG) and a silicone-containing component.

It is common to use a cross-linking agent to induce cross-linking of a polymer in preparing a hydrogel by using a polymer. The method of inducing cross-linking of a polymer by using a cross-linking agent may have the problems that, since the cross-linking agent mediates the bonding between polymers or within the polymer, the cross-linking agent may be incorporated into the hydrogel, and the cross-linking agent may remain in the reactant in an active state due to its high concentration. In addition, the cross-linking agent remaining in the hydrogel may cause various side effects after being administered into the body. However, the inventors of the present invention confirmed that intermolecular or intramolecular cross-linking of hyaluronic acid, polyethylene glycol, and/or a silicone-containing component was induced by irradiating an electron beam to a mixed aqueous solution of hyaluronic acid, polyethylene glycol and a silicon-containing component under specific conditions to form a hydrogel. A hydrogel formed only by the binding of hyaluronic acid, polyethylene glycol and/or a silicone-containing component itself, without containing external substances such as a cross-linking agent or metal cations additionally added for physical cross-linking inside the molecules, has not been previously reported and is first disclosed herein by the inventors of the present invention.

On the other hand, all medical materials as well as polymer materials necessarily require biocompatibility, and such biocompatibility can be distinguished in two ways. Biocompatibility in a broad sense means having both desired functions and safety to a living body, and biocompatibility in a narrow sense means having biological safety to a living body, that is, non-toxicity and sterilization.

However, the biocompatible hydrogel of the present invention is formed only by intermolecular or intramolecular cross-linking of hyaluronic acid, polyethylene glycol and/or a silicone-containing component. Thus, it does not have the above-mentioned problems of the hyaluronic acid-based hydrogel prepared according to the conventional method and has the advantage of having excellent biocompatibility described above. In addition, the hydrogel of the present invention can be produced by irradiating an aqueous solution, without using any organic solvent and, thus, neither involves contamination that may occur during the preparation process, nor requires complicated processes such that it is very useful industrially.

That is, the hydrogel provided by this invention features no functional groups introduced additionally to the hyaluronic acid, polyethylene glycol and silicone-containing component. Furthermore, no additional cross-linking agents are used other than hyaluronic acid and polyethylene glycol, which directly participates in or mediates the cross-linking.

In the present invention, hyaluronic acid, which is a raw material of the biocompatible hydrogel, has a very high utilization value as a carrier for drugs due to the multifunctional functional group present in its chemical structure, and is more applicable than synthetic polymers in the field of medicine and pharmacy due to its physicochemical properties such as biocompatibility and biodegradability.

In the present invention, the hyaluronic acid encompasses both hyaluronic acid, a salt of hyaluronic acid, or a mixture of hyaluronic acid and a salt of hyaluronic acid. The hyaluronic acid salt may be at least one selected from the group consisting of sodium hyaluronate, potassium hyaluronate, calcium hyaluronate, magnesium hyaluronate, zinc hyaluronate, cobalt hyaluronate and tetrabutyl ammonium hyaluronate, but the present invention is not limited thereto.

In the present invention, the polyethylene glycol has many advantages in the field of drug delivery and tissue engineering. Typically, it has high solubility in organic solvents, is non-toxic, and has no adverse reaction to immune action, thereby exhibiting excellent biocompatibility. In addition, it can easily encapsulate and release drugs as a drug carrier and is used in the pharmaceutical formulation industry as a material approved for use in the human body by the US Food and Drug Administration. In addition, polyethylene glycol improves the biocompatibility of polymers used for blood contact among hydrophilic polymers and has the greatest effect of inhibiting protein adsorption such that many applications of polyethylene glycol as biomaterials are being made.

In the present application, the silicone-containing component is a component containing [—Si—O—] unit of at least one of a monomer, macromer and prepolymer. Preferably, total Si and bound O are present in the silicon-containing component in an amount of greater than 20% by weight, and preferably greater than 30% by weight of the total molecular weight of the silicon-containing component. The silicone-containing component may comprise polymerizable functional groups (e.g., acrylate, methacrylate, acrylamide, methacrylamide, vinyl, N-vinyl lactam, N-vinylamide and styryl functional groups), but the silicone-containing component preferably comprises no functional groups, in view of the purpose of the present invention.

Examples of a silicone-containing component useful in the present invention can be found in U.S. Pat. Nos. 3,808,178, 4,120,570, 4,136,250, 4,153,641, 4,740,533, 5,034,461 and 5,070,215, and European Patent No. 0 080 539 B1, which references describe many examples of the silicone-containing component.

In the present invention, the silicone-containing component may comprise polydimethylsiloxane, caprylylmethyl trisiloxane, octamethylcyclotetrasiloxane, decamethylcyclopentasiloxane, dimethicone, and cyclosiloxane, preferably polydimethylsiloxane, and most preferably trimethylsilyl-terminated polydimethylsiloxane having the structure of Formula 1 below, but the present invention is not limited thereto.

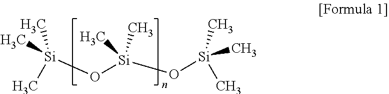

The hydrogel provided by the present invention may be produced by a method comprising the following steps:
(a) adding hyaluronic acid, polyethylene glycol and a silicone-containing component to water, thereby preparing a solution; and
(b) irradiating radiation to the solution produced in step (a) to induce cross-linking of the added substances.

The present inventors have established conditions for preparing a hydrogel formed only by intermolecular cross-linking and/or intramolecular cross-linking of hyaluronic acid, polyethylene glycol and a silicone-containing component by irradiation, through various embodiments.

According to one embodiment of the present invention, it was confirmed that irradiating an electron beam to an aqueous solution containing hyaluronic acid and a silicon-containing component did not form a hydrogel. However, it was confirmed that adding polyethylene glycol to the hyaluronic acid and silicon-containing component and irradiating an electron beam thereto under certain conditions formed a hydrogel exhibiting various physical properties.

According to another embodiment of the present invention, it was confirmed that irradiating an electron beam to an aqueous solution containing only polyethylene glycol and silicon-containing components did not induce sufficient cross-linking, thereby forming an incomplete hydrogel.

According to various embodiments of the present invention, it was confirmed that a combination of various conditions is very important to produce a hydrogel by inducing intermolecular cross-linking and/or intramolecular cross-linking of hyaluronic acid, polyethylene glycol and a silicone-containing component by irradiation. Specifically, it was confirmed that, when the molecular weight/concentration of hyaluronic acid, polyethylene glycol or the silicone-containing compound, and the amount of energy irradiation did not satisfy certain conditions, a hydrogel was not formed. In addition, it was confirmed that a hydrogel exhibiting various physical properties could be produced by appropriately adjusting such conditions.

In step (a) of the present invention, polyethylene glycol having a molecular weight of 15 to 50 kDa, preferably 15 to 40 kDa, and, most preferably a molecular weight of 20 to 35 kDa, may be used.

Using polyethylene glycol having a molecular weight of less than 15 kDa may cause that problem that a hydrogel is not formed by electron beam irradiation. Using greater than 40 kDa of polyethylene glycol may cause the problem that, when formed at a low radiation dose, the hydrogel may not have a perfect shape, and excessive bubbles or cracking may occur inside the hydrogel. In addition, when too large PEG having a molecular weight of 40 kDa or more is injected into the body, it may show reduced biodegradability and remain in the body for a very long time to cause problems.

In addition, in step (a) of the present invention, the polyethylene glycol may be added to water at a concentration of 0.1 to 3% (w/v), preferably 0.1 to 2% (w/v), more preferably 0.5 to 1.5% (w/v), and most preferably within 0.5 to 1.0% (w/v).

There is a limitation in that, when the concentration of polyethylene glycol is low, the cross-linking reaction is not easily induced such that a hydrogel is not formed; and, when the concentration is too high, only the cross-linking reaction between the polyethylene glycol strands dominates such that the hydrogel and the remaining solution coexist, that is, the three components are not uniformly cross-linked to form a gel, and a cross-linking reaction between only some components proceeds.

In step (a) of the present invention, hyaluronic acid having a molecular weight of 50 to 3000 kDa, preferably 70 to 2700 kDa, and most preferably a molecular weight of 100 to 2500 kDa, may be used.

There may be the problem that, if the molecular weight of hyaluronic acid is out of range and too small, a uniform gel is not made, and, if the molecular weight is too large, a gel is not made.

In addition, in step (a) of the present invention, hyaluronic acid may be added to water at a concentration of 0.05 to 3% (w/v), preferably 0.1 to 2% (w/v), more preferably 0.5 to 1.5% (w/v), and most preferably a 0.5 to 1.0% (w/v).

There is a limitation in that, if the concentration of hyaluronic acid is too high, it is difficult to form a hydrogel by electron beam irradiation, and the hydrogel may not be formed; as the concentration increases, the solubility of hyaluronic acid decreases, making it difficult to prepare a sample, which may cause problems in the preparation process; and, if the concentration of hyaluronic acid is too low, the properties of the hydrogel may not be well exhibited in the subsequent use of the hydrogel.

According to one embodiment of the present invention, it was confirmed that, when the concentration of hyaluronic acid in the aqueous solution used for preparing the hydrogel is higher than the concentration of polyethylene glycol, the viscosity of the resulting hydrogel is lowered and the adhesion is improved. Conversely, it was confirmed that, when the concentration of hyaluronic acid in the aqueous solution used for preparing the hydrogel is lower than the concentration of polyethylene glycol, the resulting hydrogel has high viscosity and low adhesion.

Therefore, a hydrogel exhibiting desired viscosity and adhesion may be produced by adjusting the concentrations of hyaluronic acid and polyethylene glycol in the aqueous solution in step (a).

In step (a) of the present invention, a silicone-containing component having a molecular weight of 100 to 10000 Da, preferably 200 to 10000 Da, and most preferably 200 to 9000 Da, may be used.

If the molecular weight of the silicone-containing component is less than 100 Da, a hydrogel may not be formed by electron beam irradiation, and, if the molecular weight is greater than 10000 Da, the transparency of the resulting hydrogel may be reduced.

If the molecular weight of silicon exceeds the above-mentioned range in preparing an aqueous solution before electron beam irradiation, the silicone-containing component does not mix well with hyaluronic acid and polyethylene glycol, and, even after electron beam irradiation, they may not form a hydrogel together and be separated.

In addition, in step (a) of the present invention, the silicon-containing component may be added to water at a concentration of 0.1 to 3% (w/v), preferably 0.1 to 2% (w/v), more preferably, 0.5 to 1.5% (w/v), and most preferably 0.5 to 1.0% (w/v).

If the concentration of silicon exceeds the above-mentioned range in preparing an aqueous solution before electron beam irradiation, the silicone-containing component does not mix well with hyaluronic acid and polyethylene glycol, and, even after electron beam irradiation, they may not form a hydrogel together and be separated.

A person skilled in the art may adjust conditions of the molecular weight/concentration of the hyaluronic acid, polyethylene glycol and silicone-containing component used in step (a) of the present invention such that the present invention exhibits desirable physical properties depending on the purpose for which the hydrogel is to be used.

For example, the hydrogel to be used as a wound dressing is preferably transparent, has high viscoelasticity, and exhibits excellent adhesion. To this end, in step (a), an aqueous solution comprising 2000 to 3000 kDa of hyaluronic acid at a concentration of 0.01 to 0.5% (w/v), 25 to 40 kDa of polyethylene glycol at a concentration of 0.5 to 1.0% (w/v), and 100 to 1000 Da of a silicon-containing component at a concentration of 0.1 to 0.5% (w/v) may be preferably used.

On the other hand, step (b) of the present invention is irradiating the solution produced in step (a) with radiation to induce cross-linking of the added substances.

The hydrogel molded by irradiation has the advantage that it has no residual toxicity present in hydrogels prepared by chemical methods and can achieve both cross-linking and a sterilization effect. At this time, the radiation to be used may be at least one selected from the group consisting of gamma rays, ultraviolet rays, X-rays, and electron beams, and may preferably be electron beams.

According to one embodiment of the present invention, it was confirmed that the radiation dose and/or energy intensity of the radiation irradiated to form the hydrogel in step (b) may vary depending on the molecular weight/concentration of the hyaluronic acid, polyethylene glycol and silicone-containing component used in step (a). In addition, even under conditions in which a hydrogel is formed, physical properties of the hydrogel may vary depending on the radiation dose and/or energy intensity of the irradiated radiation.

The dose of radiation irradiated in step (b) of the present invention may be preferably 0.5 to 300 kGy, more preferably 2 to 300 kGy, and most preferably 5 to 150 kGy, but the present invention is not limited thereto. If the irradiation dose is less than 0.5 kGy, sufficient cross-linking may not occur such that the formation of a hydrogel may be incomplete, and, if the radiation dose exceeds 300 kGy, bubbles may be generated inside the hydrogel.

In addition, the energy intensity of the radiation irradiated in step (b) may be 0.5 to 20 MeV, preferably 1 to 10 MeV, more preferably 1 to 5 MeV, and most preferably 1 to 2.5 MeV.

If the energy intensity of the radiation is low, a hydrogel may not be formed. In addition, if the energy intensity of the radiation is too high, the shape of the formed hydrogel may not be intact, bubbles may be formed inside the hydrogel, or the hydrogel may be cracked.

Specific examples of the conditions for preparing the hydrogel provided in the present invention are presented in the embodiments of the present invention.

The present invention also provides a method for preparing a hydrogel formed by inter-molecular cross-linking, intra-molecular cross-linking, or only in-molecular and intra-molecular cross-linking of hyaluronic acid, polyethylene glycol (PEG) and a silicone-containing component, the method comprising the following steps:
(a) adding hyaluronic acid, polyethylene glycol and a silicone-containing component to water, thereby preparing a solution; and
(b) irradiating radiation to the solution produced in step (a) to induce cross-linking of the added substances.

A detailed description of each step of the preparing method may be applied in the same manner as described above.

The present invention also provides a cell delivery system, drug delivery system, anti-adhesion agent, cell scaffold, dental filler, orthopedic filler, wound dressing (in a sheet type, gel type, spray type, cream type, etc.) or dermal filler, comprising the hydrogel.

According to one embodiment of the present invention, it was confirmed that the wound dressing made of the hydrogel according to the present invention has excellent adhesion to the wounded area, as compared to a commercial wound dressing, and significantly reduces the formation of scars in the wound healing process. This means that various endogenous wound repair factors secreted from wounds are absorbed/retained due to the excellent water-containing property of hyaluronic acid contained in the hydrogel such that the wound dressing exhibits self-healing effects, and oxygen required during the wound healing process was smoothly supplied due to the excellent oxygen permeability of the silicon-containing component.

The term “wound” of the present invention refers to a state in which the continuity of tissue is destroyed by external pressure. Wounds comprise abrasions, bruises, lacerations, and knife cuts.

The present invention can provide a hydrogel that satisfies various physical properties such as viscoelasticity and adhesiveness by changing the conditions for preparation within the above-mentioned ranges according to the intended use. In addition, since no chemical cross-linking agent and organic chemicals are used during the preparation process, the hydrogel has excellent biocompatibility and can be used for various purposes.

Biocompatible hydrogels are used in various ways, e.g., as cell carriers, drug delivery systems, anti-adhesion agents, cell scaffolds, dental fillers, orthopedic fillers, wound dressings (in a sheet type, gel type, spray type, cream type, etc.) or dermal fillers. Since research thereon is also being actively conducted in the art, it is obvious to a person skilled in the art that the hydrogel provided in the present invention can also be used for the above-mentioned purposes.

The cell delivery system, drug delivery system, anti-adhesion agent, cell scaffold, dental filler, orthopedic filler, wound dressing (in a sheet type, gel type, spray type, cream type, etc.) or dermal filler provided in the present invention may further comprise various conventional additives in addition to the hydrogel. The types of such additives may comprise, for example, dyes, colored pigments, vegetable oils, thickeners, pH adjusters, osmotic pressure regulators, vitamins, antioxidants, inorganic salts, preservatives, solubilizers, isotonic agents, suspending agents, emulsifiers, stabilizers, anesthetics, disinfectants, wound healing agents, and the like, but the present invention is not limited thereto.

The present invention also provides a composition for application to the wounded area of skin, comprising the hydrogel as an active ingredient.

The composition for skin application to the wounded area of skin may further comprise a known drug, disinfectant, etc. that can help heal the wound, and may be formulated as a wound dressing and used as a wound dressing in a sheet type, gel type, spray type or cream type.

The present invention provides the use of the hydrogel for preparing an agent for application to the wounded area of skin.

The present invention provides a method of treating the wounded area of skin by applying an effective amount of a composition comprising the hydrogel as an active ingredient to the skin of a subject in need thereof.

The term “effective amount” of the present invention refers to an amount that exhibits an effect of improving, treating, detecting, diagnosing of a wound, or inhibiting or reducing the progression of a wound, when administered to a subject. In addition, the term “individual” may be an animal, preferably a mammal, particularly an animal comprising a human being, and may also be a cell, tissue, or organ derived from an animal. The subject may be a patient in need of the effect.

The term “treatment” of the present invention comprehensively refers to improving wounded area or a symptom caused by a wound, may comprise curing, substantially preventing, or improving the condition of the wound, and comprises mitigating, curing, or preventing one or most of the symptoms caused by the disease, but the present invention is not limited thereto.

As used herein, the term “comprising” is used in the same sense as “including” or “characterized by.” The composition or method according to the present invention does not exclude additional components or method steps not specifically mentioned. In addition, the term “consisting of” refers to excluding additional elements, steps or components not separately described. The term “essentially consisting of” means that, in addition to the described materials or steps, materials or steps that do not substantially affect the basic characteristics thereof may be contained in the scope of a composition or method.

### Advantageous Effects of Invention

The hydrogel of the present invention is prepared by inducing intermolecular and/or intramolecular cross-linking of hyaluronic acid, polyethylene glycol and a silicone-containing component through electron beams. Therefore, there is no risk of toxicity problems in the human body due to incorporation of organic solvents or cross-linking agents. In addition, since no separate purification process is required during the preparation process, mass production is possible with only short electron beam irradiation, and the hydrogel of the present invention is also very excellent in terms of productivity. In addition, the hydrogel of the present invention has very excellent biocompatibility and, thus, can be very useful for the development of cell delivery systems, drug delivery systems, anti-adhesion agents, cell scaffolds, dental fillers, orthopedic fillers, wound dressings or dermal fillers.

### BRIEF DESCRIPTION OF DRAWINGS

FIG. 1 shows the results of visually observing whether a hydrogel is generated after irradiating electron beams to an aqueous solution of 100 kDa of 1% hyaluronic acid, various molecular weights of 1% PEG, and various molecular weights of 1% silicone.

FIG. 2 shows the results of visually observing whether a hydrogel is generated after irradiating electron beams to an aqueous solution of 1200 kDa of 1% hyaluronic acid, various molecular weights of 1% PEG, and various molecular weights of 1% silicone.

FIG. 3 shows the results of visually observing whether a hydrogel is generated after irradiating electron beams to an aqueous solution of 100 kDa of 1% hyaluronic acid, 35 kDa of 1% PEG, and various molecular weights of 1% silicone.

FIG. 4 shows the results of visually observing whether a hydrogel is generated after irradiating electron beams to an aqueous solution of 2500 kDa of 1% hyaluronic acid, various molecular weights of 1% PEG, and various molecular weights of 1% silicone.

FIG. 5 shows the results of visually observing whether a hydrogel is generated after irradiating electron beams to an aqueous solution of 100 kDa of 1% hyaluronic acid, 35 kDa of 1% PEG, and 9000 Da of 1% silicone.

FIG. 6 shows the results of visually observing whether a hydrogel is generated after irradiating electron beams to an aqueous solution of 2500 kDa of 1% hyaluronic acid, 35 kDa of 1% or 0.5% of PEG, and 237 Da or 9000 Da of 1% or 0.5% silicone.

FIG. 7 shows the results of visually observing whether a hydrogel is generated after irradiating electron beams to an aqueous solution of 2500 kDa of 0.5% hyaluronic acid, 35 kDa of 1% PEG, and 237 Da of 0.5% silicone.

FIG. 8 shows an experimental process in a wounded animal model.

FIG. 9 shows the results of visually observing the wounded area of the wounded animal models over time after they are untreated (control) or treated with Medifoam (positive control) and hydrogel (HA-PEG-Si gel) according to the present invention.

FIG. 10 shows the results of visually observing whether a hydrogel is generated by placing an aqueous solution of 2500 kDa of 1% hyaluronic acid, 35 kDa of 1% PEG, and 237 Da of 0.5% silicone in a large-capacity container and then irradiating electron beam thereto.

FIG. 11 shows the results of evaluating the swelling index of the lyophilized hydrogel according to an embodiment of the present invention.

FIG. 12 shows photographs comparing before and after the hydration of a lyophilized hydrogel according to an embodiment of the present invention.

FIG. 13 shows the results of spectroscopic structural analysis through UV-Vis spectrum of a hydrogel according to an embodiment of the present invention (EB: electron beam irradiation).

FIG. 14 shows the results of structural analysis by FT-IR spectroscopy of a hydrogel according to an embodiment of the present invention (Before EB: before electron beam irradiation; and After EB: after electron beam irradiation).

FIG. 15 shows the results of visual observation using an electron microscope (SEM) of a hydrogel according to an embodiment of the present invention.

### MODE FOR INVENTION

Hereinafter, the present invention will be described in detail by the following embodiments. However, the following embodiments are only for illustrating the present invention, and the present invention is not limited thereto.

### Example 1: Preparation of a Hyaluronic Acid (HA)-Polyethylene Glycol (PEG)-Silicone Hydrogel Through Electron Beam Irradiation

A screening experiment was carried out to see under what conditions a hydrogel is made by varying the molecular weight of HA, PEG, and silicone.

HA having three molecular weights of 100 kDa, 1200 kDa and 2500 kDa, PEG having five molecular weights of 1 kDa, 3 kDa, 10 kDa, 20 kDa and 35 kDa, and silicone (i.e., trimethylsiloxy terminated polydimethylsiloxane) having four molecular weights of 237 kDa, 1250 kDa, 4000 kDa and 9000 kDa were used.

At this time, the irradiation dose of the electron beam used was fixed at 2.5 MeV and 10 kGy, and each of the above-mentioned substances was prepared as an aqueous solution having a concentration of 1% (w/v), and the electron beam was irradiated thereto.

First, in summarizing the results of the conditions in which 100 kDa HA was used, it was confirmed that, when the molecular weight of PEG was less than 20 kDa, a hydrogel was not formed regardless of the molecular weight of silicone; and the viscoelasticity was lowered, as compared to that before electron beam irradiation (see Table 1).

TABLE 1
100 kDa of 1% HA + 1% PEG + 1% Silicone/2.5 MeV, 10 kGy

| | | PEG (kDa) | | | | |
|---|---|---|---|---|---|---|
| HA 100 kDa | | 1 | 3 | 10 | 20 | 35 |
| Silicone | 237 | X | X | X | ◯ | ◯ |
| (Da) | 1250 | X | X | X | ◯ | ◯ |
| | 4000 | X | X | X | ◯ | ◯ |
| | 9000 | X | X | X | ◯ | ◯ |

(◯: hydrogel formed, X: hydrogel not formed)

It was confirmed that, when the molecular weight of PEG was 20 kDa or more, a hydrogel was formed regardless of the molecular weight of silicone, but the hydrogel tended to become opaque as the molecular weight of silicone increased; and, when the hydrogel to which 35 kDa PEG was added, the hydrogel did not break easily and agglomerated to form an elastic mass.

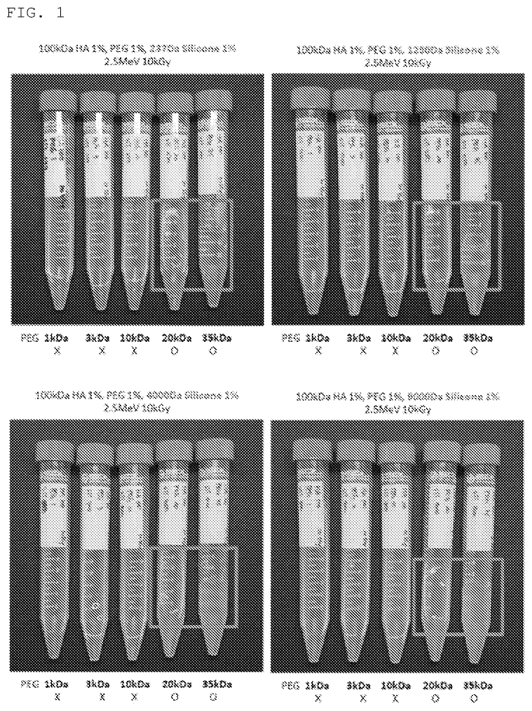

It was confirmed that the hydrogel prepared under all conditions was easily attached to the conical tube wall and easily detached, and such characteristics were not affected by the molecular weight of silicone (see FIG. 1).

Next, in summarizing the results under the conditions in which 1200 kDa HA was used, it was confirmed that, when the molecular weight of PEG was less than 20 kDa, a hydrogel was not formed regardless of the molecular weight of silicone, as shown in the results obtained by using 100 kDa HA, and the tendency and characteristics were the same as those of 100 kDa HA (see Table 2).

TABLE 2
1200 kDa of 1% HA + 1% PEG + 1% Silicone/2.5 MeV, 10 kGY

| | | PEG (kDa) | | | | |
|---|---|---|---|---|---|---|
| HA 120 kDa | | 1 | 3 | 10 | 20 | 35 |
| Silicone | 237 | X | X | X | ◯ | ◯ |
| (Da) | 1250 | X | X | X | ◯ | ◯ |
| | 4000 | X | X | X | ◯ | ◯ |
| | 9000 | X | X | X | ◯ | ◯ |

(◯: hydrogel formed, X: hydrogel not formed)

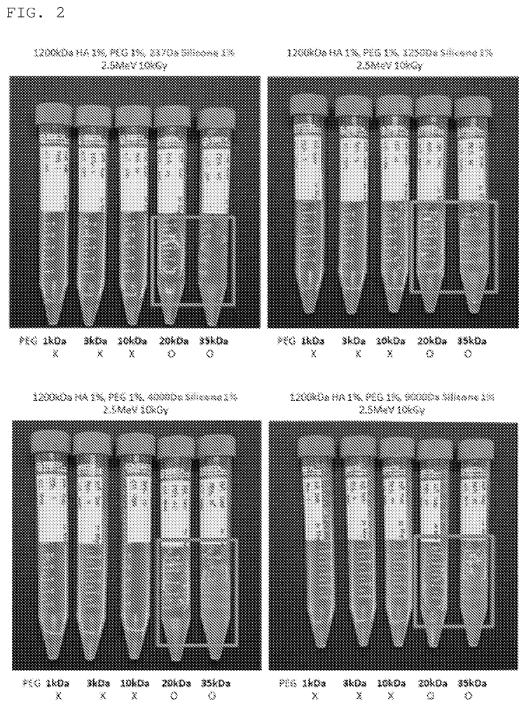

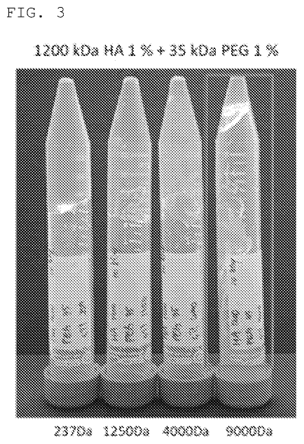

However, the hydrogels to which 35 kDa PEG and 9000 Da silicone were added had greater flow properties and exhibited properties closer to liquids, as compared to the hydrogels made from silicones having other molecular weights (see FIGS. 2 and 3).

Next, in summarizing the results for the conditions in which 2500 kDa HA was used, it was confirmed that, when the molecular weight of PEG was less than 20 kDa, a hydrogel was not formed regardless of the molecular weight of silicone, like the results obtained by using 100 kDa and 1200 kDa HA, and whether or not a hydrogel was formed was the same as that when 100 kDa and 1200 kDa HA were used (see Table 3 and FIG. 4).

TABLE 3
2500 kDa of 1% HA + 1% PEG + 1% Silicone/2.5 MeV, 10 kGy

| | | PEG (kDa) | | | | |
|---|---|---|---|---|---|---|
| HA 2500 kDa | | 1 | 3 | 10 | 20 | 35 |
| Silicone | 237 | X | X | X | ◯ | ◯ |
| (Da) | 1250 | X | X | X | ◯ | ◯ |
| | 4000 | X | X | X | ◯ | ◯ |
| | 9000 | X | X | X | ◯ | ◯ |

Through the above-mentioned experimental results, it was confirmed that, when preparing a hydrogel by irradiating an electron beam to an aqueous solution containing 1% (w/v) of each of HA, PEG and silicone, the molecular weight range of PEG greatly affects the formation of a hydrogel.

Next, three components having a high molecular weight were used to check whether a hydrogel was generated at the same various electron beam irradiation doses.
Conditions for the aqueous solution: 2500 kDa of 1% HA+35 kDa of 1% PEG+9000 Da of 1% Silicone
Conditions for the electron beam irradiation: 2.5 MeV, 10 kGy, 50 kGy, 100 kGy and 200 kGy

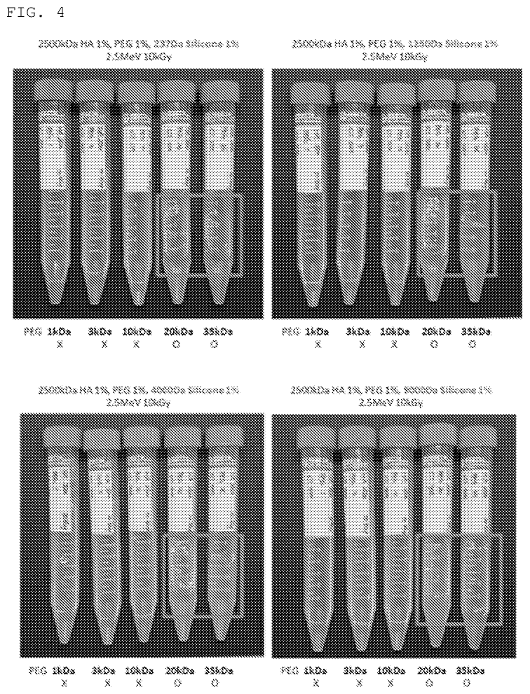

As a result, as shown in FIG. 5, it was confirmed that a hydrogel was formed even at the electron beam irradiation dose of 10 kGy. In addition, it was confirmed that the hydrogel comprising 9000 Da of silicone, which is a higher molecular weight of silicone, became more opaque, as compared to the molecular weight of 237 Da. On the other hand, it was confirmed that bubbles in the resulting hydrogel were noticeably increased at the molecular weight of 200 kGy.

Next, in order to check the difference in the formation and characteristics of the hydrogel depending on the concentration of each of the HA, PEG and silicone, the experiment was conducted while changing the concentration of the aqueous solution of each of HA, PEG and silicone to 0.5% or 1%.

At this time, 2500 kDa of HA and 35 kDa of PEG were constantly used, silicone having two molecular weights of 237 Da and 9000 Da were used, and electron beams of 2.5 MeV and 10 kGy were irradiated to carry out the experiment.

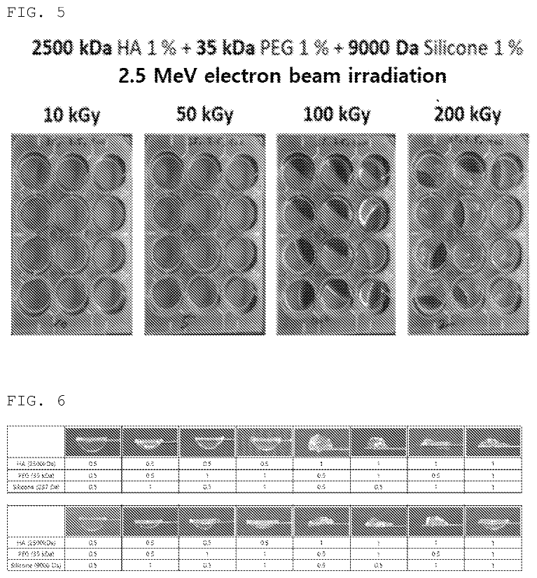

The results thereon are shown in FIG. 6.

As shown in FIG. 6, it was confirmed that a hydrogel was formed in all conditions for preparation. Specifically, the hydrogel made when the concentration of HA was higher than the concentration of PEG showed high adhesion and low viscoelasticity (or shape retention), and, conversely, the hydrogel made when the concentration of HA was lower than that of PEG exhibited low adhesion and strong viscoelasticity (or shape retention).

It was confirmed that, when the concentrations of HA and PEG were the same, both adhesion and viscoelasticity (or shape retention) were maintained to some extent, but the disc shape was not maintained intact.

It was confirmed that, the higher the concentration of silicone, the higher the adhesiveness of the hydrogel, and the higher the molecular weight of the silicone, the darker the color of the hydrogel.

### Example 2: Evaluation on Efficacy of a HA+PEG+Silicone Hydrogel as a Wound Dressing

An experiment was carried out to evaluate the efficacy of the wound dressing of the hydrogel prepared under the conditions of 2500 kDa of 0.5% HA+35 kDa of 1% PEG+237 Da of 0.5% silicone (2.5 MeV, 10 kGy) in which the shape of the disc was well maintained, the hardest physical properties were exhibited, and the transparency was high in the above-described Example 1.

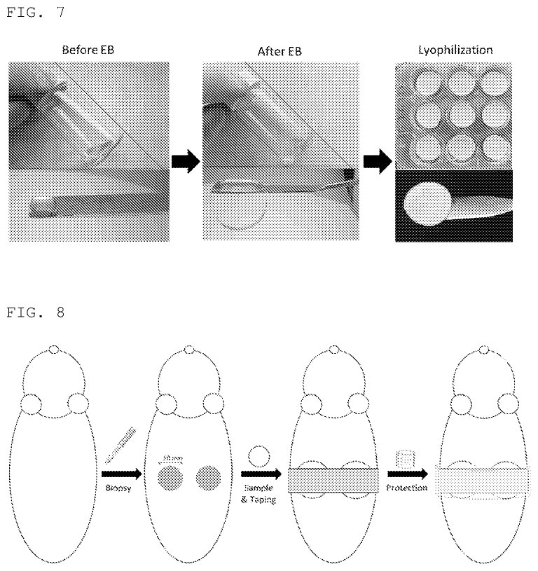

The prepared hydrogel was additionally subjected to a lyophilization process to be used as a wound dressing. Even after lyophilization, the shape of the disk was maintained, and after lyophilization, the hydrogel was easily separated from the electron beam irradiation reactor (see FIG. 7).

An animal model was created by using BALB/c mice to evaluate the efficacy of the wound dressing of the hydrogel.

Under gas anesthesia, hair on the back of BALB/c mice was cleanly depilated, and a wound was made on each of the left and right sides by using a biopsy punch with a diameter of 10 mm, and a lyophilized hydrogel was placed on both the left and right wounds, followed by dressing the wounds by using a medical paper tape.

A 50 ml tube was cut to a length of 2 cm to additionally cover the dressings in order to prevent the mouse from chewing the tapes, and the lyophilized hydrogel was replaced once every 3 days to monitor the size of the wounds (see FIG. 8).

At this time, the group for whom Medifoam, a commercially available wound dressing, was replaced with the same size as the hydrogel prepared in the present invention once every 3 days, was added, and the group treated with nothing on the wounds was added as a control group. A total of such three groups were monitored for 27 days, and the efficacies of their wound dressing were compared and evaluated.

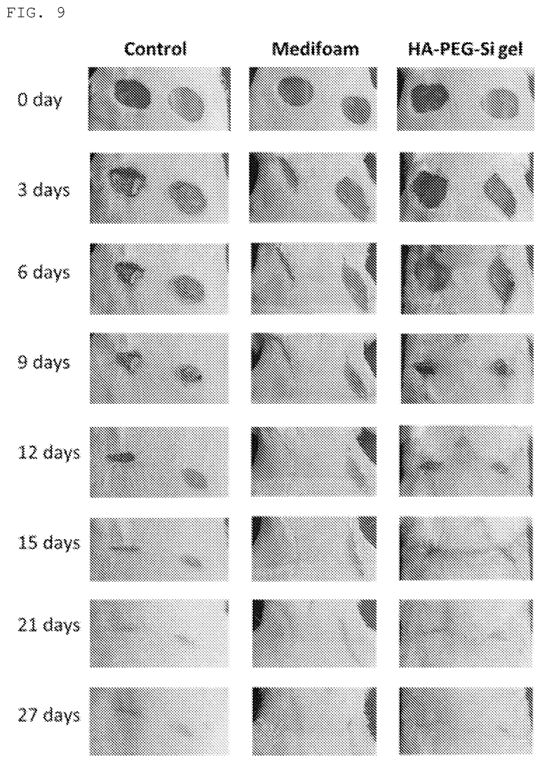

The results thereon are shown in FIG. 9.

As shown in FIG. 9, there was no significant difference in terms of wound healing or skin regeneration speed in the three groups. However, it was confirmed that litter or foreign substances easily adhered to the wounds of the control group, which caused a problem in that the wounds could not be protected, and the risk of infection could easily occur.

It was confirmed that the wound area of the group treated with Medifoam could be more protected, as compared to the control group, but the Medifoam product did not easily adhered to the wound area due to its nature, and the wound areas were in contact with each other, leaving deep scars.

In the case of the group treated with the freeze-dried hydrogel (HA-PEG-Silicone) prepared in the present invention, it was confirmed that the wounded area could be protected, and, thanks to the property of easily adhering to the wounded area, the group showed significantly reduced side effects shown in the group treated with Mediform due to the feature that the wounded areas are in contact with each other. As a result of monitoring all groups for 27 days, scars remained the smallest in this group.

### Example 3: Preparation of Large-Capacity Hydrogels for Mass Production

In order to confirm that the hydrogel prepared in a small amount in Example 1 can also be produced on a large scale, additional experiments were carried out by increasing the volume and area of the sample of the electron beam irradiation reactor.

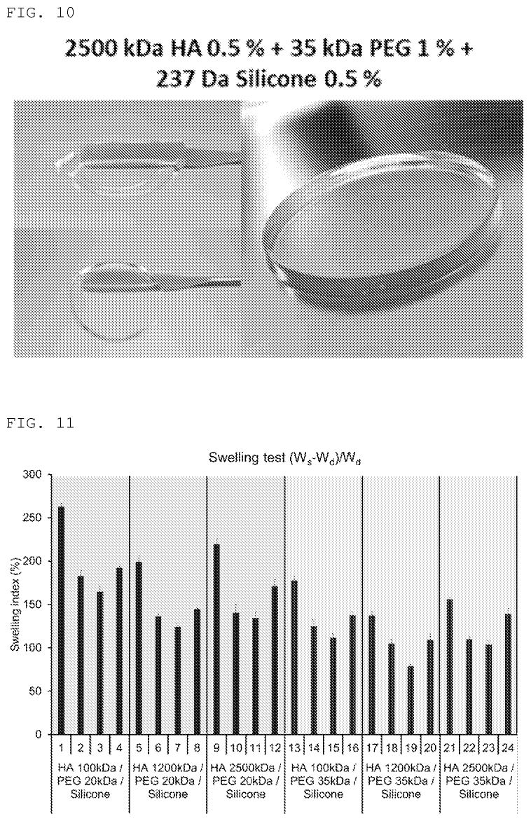

When the experiment was carried out by doubling the capacity of the sample in the electron beam irradiation reactor (2.5 MeV 10 kGy) previously used for the electron beam irradiation experiment, it was confirmed that, even though the sample capacity was doubled, the resulting hydrogel had properties very similar to those of the existing hydrogel (see FIG. 10).

In addition, the experiment was carried out by using a reactor (3.5 cm in diameter) with a larger area than that of the reactor (2.5 cm in diameter) used as the existing electron beam irradiation reactor, and, when the electron beam irradiation was performed, it was confirmed that, even though the area of the reactor was increased, the resulting hydrogel exhibited the same physical properties as those of the hydrogel for which the reactor (2.5 cm in diameter) was used.

### Example 4: Evaluation of the Swelling Index of a HA+PEG+Silicone Hydrogel

HA+PEG+silicone hydrogels having various compositions shown in Table 4 below were prepared in the same manner as in Example 1, and then their swelling indexes were evaluated.

TABLE 4

| No. | HA 1% | | PEG 1% | Silicone 1% |
|---|---|---|---|---|
| 1 | 100 | kDA | 20 kDA | 237 |
| 2 | | | | 1250 |
| 3 | | | | 4000 |
| 4 | | | | 9000 |
| 5 | 1.2 | MDa | 20 kDa | 237 |
| 6 | | | | 1250 |
| 7 | | | | 4000 |
| 8 | | | | 9000 |
| 9 | 2.5 | MDa | 20 kDa | 237 |
| 10 | | | | 1250 |
| 11 | | | | 4000 |
| 12 | | | | 9000 |
| 13 | 100 | kDa | 35 kDa | 237 |
| 14 | | | | 1250 |
| 15 | | | | 4000 |
| 16 | | | | 9000 |
| 17 | 1.2 | MDa | 35 kDa | 237 |
| 18 | | | | 1250 |
| 19 | | | | 4000 |
| 20 | | | | 9000 |
| 21 | 2.5 | MDa | 35 kDa | 237 |
| 22 | | | | 1250 |
| 23 | | | | 4000 |
| 24 | | | | 9000 |

The swelling index was calculated by the formula below.

Swelling Index (%)=(Ws−Wd)/Wd*100
Ws: a weight of a hydrogel containing water, and Wd: a weight of a dried hydrogel

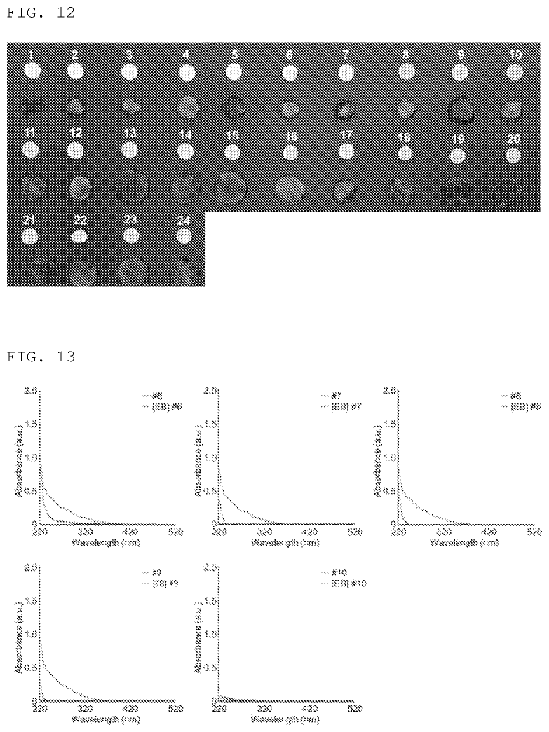

The results of the swelling index of each hydrogel prepared with the compositions of Table 4 are shown in FIG. 11, and photographs comparing before and after the hydration of the lyophilized hydrogel are shown in FIG. 12.

As shown in FIG. 11, it was confirmed that:
under the conditions in which PEG and silicone had the same molecular weight, the swelling index according to the molecular weight of HA was higher when HA was 100 kDa than that when HA was 2500 kDa or 1200 kDa;
when PEG and silicone had the same molecular weight, the swelling index according to the molecular weight of PEG was higher when PEG was 20 kDa than that when PEG was 35 kDa;
when the hydrogel was formed with only 20 kDa of PEG and 35 kDa of PEG, there was no significant difference in the swelling index, but when the hydrogel was mixed with HA, the swelling index was significantly higher when PEG was 20 kDa than that when it was 35 kDa;
Under the conditions in which HA and PEG had the same molecular weight, the swelling index according to the molecular weight of silicone was highest when silicone was 237 Da; and
the swelling index decreased as the molecular weight of silicone increased to 1250 Da and 4000 Da, but the swelling index increased again when silicone was 9000 Da.

### Example 5: Structural Analysis of HA+PEG+Silicone Hydrogels

After preparing hydrogels having various compositions shown in Table 5 below in the same manner as in Example 1, their structures were analyzed by UV-Vis, FT-IR and SEM.

TABLE 5

| No. | HA 1% | | Silicone 1% | PEG 1% |
|---|---|---|---|---|
| 6 | 100 | kDa | 237 Da | 35 kDa |
| 7 | 2.5 | Mda | 237 Da | 35 kDa |
| 8 | 100 | kDa | — | 35 kDa |
| 9 | 2.5 | Mda | — | 35 kDa |

| | | | |
|---|---|---|---|
| 10 | — | — | 35 kDa |

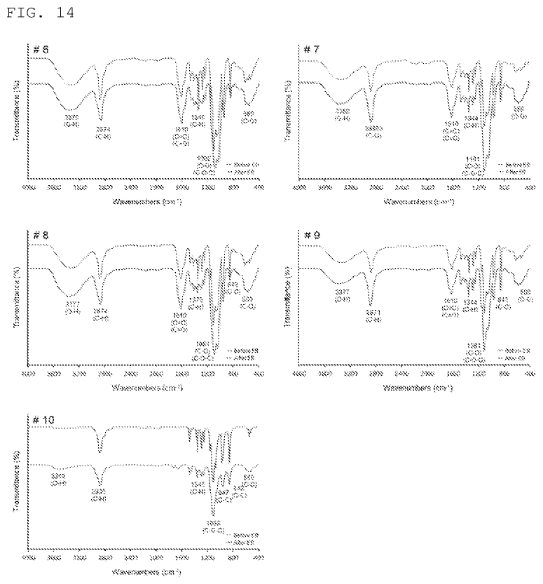

As a result of spectroscopic structural analysis through the UV-Vis spectrum, as shown in FIG. 13, it was confirmed that, except for the hydrogel consisting of only PEG, hydrogel Nos. 6, 7, 8, and 9 containing hyaluronic acid showed an increase in absorbance up to the UV-B and A regions, but no absorption in the visible band after 400 nm was observed; and the difference in absorbance of the hydrogel consisting of only PEG before and after electron beam irradiation was insignificant.

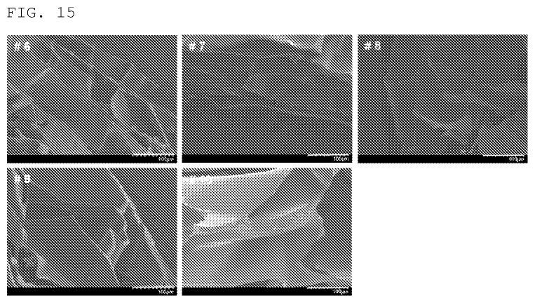

As a result of structural analysis through FT-IR spectroscopy, as shown in FIG. 14, it was confirmed that an increase in the 560 cm⁻¹ peak was observed in all samples after forming a hydrogel by electron beam irradiation, which was considered to be the result of increased bending of the C—O bond due to the cross-linking of PEG itself. On the other hand, the peak near 843 cm⁻¹ decreased in size after electron beam irradiation, which was considered that the skeletal vibration of the C—C bond due to self-cross-linking was reduced.

In the case of PEG, after formation of a hydrogel by electron beam irradiation, an O—H stretching peak at 3369 cm⁻¹ was newly observed due to the improvement of the function of the hydrogel. According to the self-cross-linking reaction, the C—H bending at 1345 cm⁻¹ and the C—C skeletal vibration bands at 842 and 947 cm⁻¹ decreased. A typical triplet splitting pattern peak due to C—O stretching vibrational stretching was observed at 1093 cm⁻¹ in PEG before electron beam irradiation.

As a result of confirming the network structure formation using an electron microscope (SEM), as shown in FIG. 15, a lamellar layered structure was observed in all samples. A very thin plate-like structure was observed in sample Nos. 6 and 7 containing silicon, and sample Nos. 8 and 9 containing only HA and PEG had wider interlayer spacing than those of the samples containing silicon. A porous material closer to a honeycomb structure, rather than a lamellar structure, was observed in sample No. 10 consisting of only PEG.

### Comparative Example 1: Preparation of HA+Silicone Hydrogels by Electron Beam Irradiation

When each of 237 Da and 9000 Da of 1% silicone was added to 2500 kDa of a 1% hyaluronic acid aqueous solution and then irradiated with a 2.5 MeV 10 kGy electron beam, a hydrogel was not produced in either composition. Therefore, it was confirmed that polyethylene glycol is essential to produce a hydrogel by using a hyaluronic acid and silicone.

### Comparative Example 2: Preparation of PEG+Silicone Hydrogels by Electron Beam Irradiation

When each of 237 Da and 9000 Da of 1% silicon was added to 35 kDa of a 1% polyethylene glycol aqueous solution and then irradiated with a 2.5 MeV 10 kGy electron beam, a hydrogel was partially produced in either composition. Unlike the hydrogel to which hyaluronic acid was added, the hydrogel was not made on the entire area of the container, and a small circular gel was made in a contracted form only in the center and the solution was left around. As a result, it was confirmed that, when only polyethylene glycol and silicone were used, 100% hydrogel was not made; and a hyaluronic acid is essential to prepare a hydrogel having a uniform composition.

### Comparative Example 3: Preparation of HA+PEG+Collagen Hydrogels by Electron Beam Irradiation

After adding 1% collagen to an aqueous solution of 2500 kDa of 1% hyaluronic acid+35 kDa of 1% PEG, a hydrogel was prepared by irradiation with an electron beam. When collagen was added to the hyaluronic acid aqueous solution, a white precipitate was formed, and, even when the electron beam was irradiated, a hydrogel was not produced and the white precipitate did not disappear either. Although 35 kDa polyethylene glycol was first added to 2500 kDa the hyaluronic acid aqueous solution before adding collagen by another preparation method, the white precipitate was formed by adding collagen, and a hydrogel was not prepared even when irradiated with electron beams.

### INDUSTRIAL APPLICABILITY

The hydrogel of the present invention is prepared by inducing intermolecular and/or intramolecular cross-linking of hyaluronic acid, polyethylene glycol and a silicone-containing component through electron beams. Therefore, there is no risk of toxicity problems in the human body due to incorporation of organic solvents or cross-linking agents. In addition, since no separate purification process is required during the preparation process, mass production is possible with only short electron beam irradiation, and the hydrogel of the present invention is also very excellent in terms of productivity. In addition, the hydrogel of the present invention has very excellent biocompatibility and, thus, can be very useful for the development of cell delivery systems, drug delivery systems, anti-adhesion agents, cell scaffolds, dental fillers, orthopedic fillers, wound dressings or dermal fillers. Therefore, the hydrogel of the present invention has a high industrial applicability.

1. A hydrogel formed only by inter-molecular cross-linking, intra-molecular cross-linking, or intermolecular and intra-molecular cross-linking of hyaluronic acid, polyethylene glycol (PEG) and a silicone-containing component.

2. The hydrogel of claim 1, wherein the intermolecular cross-linking and intra-molecular cross-linking are formed by the irradiation of radiation.

3. The hydrogel of claim 2, wherein the radiation is at least one selected from the group consisting of gamma rays, ultraviolet rays, X-rays and electron rays.

4. The hydrogel of claim 1, wherein the hydrogel is prepared by a method comprising the following steps:
(a) adding hyaluronic acid, polyethylene glycol and a silicone-containing component to water, thereby preparing a solution; and
(b) irradiating radiation to the solution produced in step (a) to induce cross-linking of the added substances.

5. The hydrogel of claim 4, wherein the polyethylene glycol has a molecular weight of 15 to 50 kDa and is added to water at a concentration of 0.1 to 3% (w/v).

6. The hydrogel of claim 4, wherein the hyaluronic acid has a molecular weight of 50 to 3000 kDa and is added to water at a concentration of 0.05 to 3% (w/v).

7. The hydrogel of claim 4, wherein the silicone-containing component is selected from the group consisting of polydimethylsiloxane, caprylylmethyl trisiloxane, octamethylcyclotetrasiloxane, decamethylcyclopentasiloxane, dimethicone and cyclosiloxane.

8. The hydrogel of claim 4, wherein the silicone-containing component has a molecular weight of 100 to 10000 Da and is added to water at a concentration of 0.1 to 3% (w/v).

9. The hydrogel of claim 4, wherein the radiation dose is 0.5 to 300 kGy.

10. The hydrogel of claim 4, wherein the energy intensity of the radiation is 0.5 to 20 MeV.

11. A method for preparing a hydrogel formed only by inter-molecular cross-linking, intra-molecular cross-linking, or intermolecular and intra-molecular cross-linking of hyaluronic acid, polyethylene glycol (PEG) and a silicone-containing component, the method comprising the following steps:
(a) adding hyaluronic acid, polyethylene glycol and a silicone-containing component to water, thereby preparing a solution; and
(b) irradiating radiation to the solution produced in step (a) to induce cross-linking of the added substances.

12. The method for preparing a hydrogel of claim 11, which comprises adjusting the viscosity of the hydrogel by changing the concentration ratio of hyaluronic acid and polyethylene glycol in step (a).

13. A cell delivery system, drug delivery system, anti-adhesion agent, cell scaffold, dental filler, orthopedic filler, dermal filler or wound dressing, comprising the hydrogel of claim 1.

14. A sheet-type, cream-type, gel-type or spray-type wound dressing comprising the hydrogel of claim 1.

15. A composition for application to the wounded area of skin comprising the hydrogel of claim 1 as an active ingredient.

16. Use of the hydrogel according to claim 1 for preparing an agent for application to the wounded area of skin.

17. A method of treating a wound by applying an effective amount of a composition comprising the hydrogel of claim 1 as an active ingredient to the skin of a subject in need thereof.
